(19) United States
(12) Patent Application Publication
(10) Pub. No.: US 2021/0238124 A1
(43) Pub. Date: Aug. 5, 2021

(54) ACETYLATED PRODRUGS FOR DELIVERY ACROSS THE BLOOD-BRAIN BARRIER

(71) Applicant: THE GENERAL HOSPITAL CORPORATION, Boston, MA (US)

(72) Inventors: David R. ELMALEH, Newton, MA (US); Timothy M. SHOUP, Franklin, MA (US)

(73) Assignee: The General Hospital Corporation, Boston, MA (US)

(21) Appl. No.: 17/049,230

(22) PCT Filed: Apr. 22, 2019
(86) PCT No.: PCT/US19/28515

§ 371 Date: Oct. 20, 2020

Related U.S. Application Data
(60) Provisional application No. 62/660,365, filed on Apr. 20, 2018.

(51) Int. Cl. C07C 233/18; C07C 219/28; C07C 217/60; A61K 51/04; A61K 51/12

(52) U.S. Cl. CPC C07C 233/18; C07C 219/28; C07B 2200/05; A61K 51/0406; A61K 51/1279; C07C 217/60

(57) ABSTRACT

The present disclosure relates to pharmaceutical compositions including a compound derived from a parent compound having a hydroxyl or amino moiety, wherein the hydroxyl in the parent compound is presented as an ester in the compound or the amino in the parent compound is presented as an amide in the compound, and their use to prevent or treat neurological disease.

### CROSS-REFERENCE TO RELATED APPLICATIONS

This application claims the benefit of U.S. Provisional Application No. 62/660,365, filed on Apr. 20, 2018. The entire teachings of the above application is incorporated herein by reference.

### BACKGROUND

The blood-brain barrier (BBB) restricts entry into the brain of most molecules from the blood. Brain uptake is possible by a few transport mechanisms: simple diffusion; diffusion via concentration equilibrium; facilitated diffusion where compounds attach to membrane protein carriers using concentration equilibrium; and an active transport, which requires energy to enable movement against concentration gradients.

While it is assumed that some small molecules are freely transported across the BBB, most therapeutics including large molecules do not cross the BBB. Indeed, the BBB is the fundamental problem hindering progress in the development of new therapeutics for brain disorders or the development of new radiopharmaceuticals to image the brain. At the same time, the BBB acts as an effective defense mechanism—the brain complex network, which controls cognition and function, is protected from toxins by the BBB.

One solution to facilitate transport of therapeutics is to disrupt the BBB. For example, osmotic disruption of the BBB by intra-arterial mannitol injection is sometimes a key step for the delivery of therapeutic drugs to brain tissue or for certain brain diseases and injuries in order to reduce pressure due to intra brain edema. Osmotic BBB opening is mediated by osmotically induced shrinkage of cerebrovascular endothelial cells and consequent reversible widening of inter-endothelial tight junctions. Osmotic treatment reduces the overall dose of drug necessary to achieve a therapeutic effect against degenerating cells, thereby reducing the potential for adverse effects on peripheral organs. However, BBB disruption for a long period of time may cause brain damage. Controlled temporary BBB disruption with mannitol can be highly variable and could impact local drug deposition.

Thus, there is a continuing need for compounds and compositions that can enter the brain across the blood-brain barrier.

### SUMMARY

Disclosed herein are compounds and compositions that provide increased brain penetration of compounds that affect specific brain functions (e.g., therapeutic neurotransmitters and ligands).

In one aspect, the invention relates to a pharmaceutical composition comprising a compound derived from a parent compound having a hydroxyl or amine moiety, and wherein the hydroxyl in the parent compound is presented as an ester (e.g., acetyl ester) or a carbonate; or the amine in the parent compound is presented as an amide (e.g., N-acetyl amine).

In another aspect, the invention relates to methods of treating or preventing a neurological disease in an organism comprising administering a compound or composition disclosed herein.

In yet another aspect, the invention relates to methods of treating or preventing a cancer in an organism comprising administering a compound or composition disclosed herein.

In still another aspect, the invention relates to methods of imaging a brain of an organism suffering for a neurological disease comprising administering a compound or composition disclosed herein.

In another embodiment, the compound disclosed herein is an acetylated prodrug of neurotransmitters or ligands specific to subclasses of neurotransmitters such as Do to D₁₆ or HTf to HTe.

In another embodiment, the compound disclosed herein is an acetylated compound that is more lipophilic (for example, as measured by the Log P of the compound) or that has a PSA (polar surface area) adjusted to a value in the range that allows brain penetration (for example, Log P=1-3; PSA=40-120 Å²).

In another embodiment, the compound disclosed herein is designed to be a prodrug or analog that allows for radiolabeling or fluorescence, for imaging purposes.

In some embodiments, the prodrug may have acetylated hydroxy and amino groups on the same molecule.

In some embodiments, the compound disclosed herein is a peptide or a protein (e.g, a monoclonal antibody) that is acetylated. In some embodiments, the acetylated protein is more lipophilic, which increases its BBB uptake.

In some embodiments, the compound disclosed herein is an acetylated nucleic acid (e.g., siRNA, miRNA). In some embodiments, the acetylated nucleic acid has enhanced BBB uptake. In some embodiments, the nucleic acid is biotinylated.

In some embodiments, the composition or formulation further comprises an excipient that disrupts the BBB (e.g., mannitol). In some embodiments, the excipient may enhance the compound's temporary brain uptake.

In other embodiment, the compounds disclosed herein are derivatized with a molecule that is recognized by brain membrane proteins, which enables diffusion or active transport.

### BRIEF DESCRIPTION OF THE DRAWINGS

FIG. 1 shows the biodistribution of 4-[¹⁸F]-fluoroinositol in normal rats.

FIG. 2 shows a MicroPET image of an invisible prostate tumor expression in mice using 4-[¹⁸F]-fluoroinositol.

FIG. 3 shows the tumor uptake (upper line) compared to normal muscle (lower line) of 4-[¹⁸F]-fluoroinositol.

FIG. 4 shows the uptake (% DPG) at 5, 30 and 60 min for the acetylated derivative 1.

FIG. 5 shows the uptake (% DPG) at 5, 30 and 60 min for derivative 2.

FIG. 6 shows the time activity curve for [¹⁸F]-fluoromannitol.

FIG. 7 shows a MicroPET image of a brain in a rat using [¹⁸F]-fluoromannitol.

FIG. 8 shows the time activity curve for D-mannitol.

FIG. 9 shows a MicroPET image of a brain in a rat using D-mannitol.

FIGS. 10A-10C show a MicroPET/CT image of a brain in a rat 10 minutes after administration of [¹⁸F]-N-[2-[3,4-Bis(acetyloxy)-6-fluorophenyl]ethyl] acetamide (F-18-6FPBA) (10A); 6-[¹⁸F]Fluorodopamine (10B); and [¹⁸F]-2,2-Dimethyl-4-[2-[(2,2-dimethyl-1-oxopropyl) amino]ethyl]-1,2-6-fluorophenylene propanate ester (F-18-6FPBPE) (10C).

FIGS. 11A-11C show a MicroPET/CT image of a brain in a rat 60 minutes after administration of F-18-6FPBA (11A); 6-[¹⁸F]Fluorodopamine (11B); and F-18-6FPBPE (11C).

FIG. 12 shows a time activity curve a for F-18-6FPBA, 6-[¹⁸F]Fluorodopamine, and F-18-6FPBPE.

### DETAILED DESCRIPTION

### General

Disclosed herein are compounds, compositions, and methods useful for improving the brain uptake of compounds with poor blood brain barrier (BBB) penetration. Not wishing to be bound by theory, the inventors discovered that using a modified form of a compound (e.g., an acetylated form) increased its effectiveness to target the brain. The inventors recognized that this modified compound provided several molecular mechanistic advantages including i) the compound was more lipophilic and, therefore, had improved BBB penetration; and ii) the compound could be administered by an alternate route (e.g., transdermal) which may improve patient compliance by reducing the possibility of missing a dose as compared to, for example, oral administration. The modified form of a compound (e.g., an acetylated form) disclosed herein may undergo an enzymatic hydrolysis by a deacetylase enzyme expressed in the brain and selectively target various brain receptors, cancer targets or neurodegenerative disease targets. For example, the high concentration of several deacetylase enzymes (e.g., histone deacetylases (HDACs)) in the brain will remove the acetyl group and allow the drug to act intra brain.

The modified compounds disclosed here improve the blood brain barrier (BBB) penetration of the parent compound; so, the compounds may be used in the treatment of disease (e.g., neurological disease) or in brain imaging.

### Definitions

The terms “a,” “an,” “the” and similar referents used in the context of describing the present invention are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any unclaimed element is essential to the practice of the invention.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group. An alkyl group with two open valences is sometimes referred to as an alkylene group, such as methylene, ethylene, propylene and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxy carbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively. As applied to heteroalkyls, “C_{x-y}” indicates that the group contains from x to y carbons and heteroatoms in the chain. As applied to carbocyclic structures, such as aryl and cycloalkyl groups, “C_{x-y}” indicates that the ring comprises x to y carbon atoms. As applied to heterocyclic structures, such as heteroaryl and heterocyclyl groups, “C_{x-y}” indicates that the ring contains from x to y carbons and heteroatoms. As applied to groups, such as aralkyl and heterocyclylalkyl groups, that have both ring and chain components, “C_{x-y}” indicates that the ring and the chain together contain from x to y carbon atoms and, as appropriate heteroatoms.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

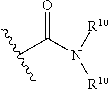

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

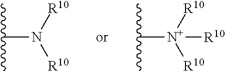

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

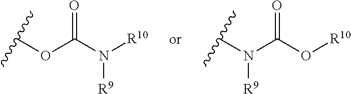

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent. In analogy with alkyl groups, heteroalkyl groups with two open valences are sometimes referred to as heteroalkylene groups. Preferably, the heteroatoms in heteroalkyl groups are selected from O and N.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the poly cycle contains from 3 to 10 atoms in the ring, preferably from 5 to 7. When a polycyclic substituent is attached through an aryl or heteroaryl ring, that substituent may be referred to herein as an aryl or heteroaryl group, while if the polycyclic substituent is attached through a cycloalkyl or heterocyclyl group, that substituent may be referred to herein as a cycloalkyl or heterocyclyl group. By way of example, a 1,2,3,4-tetrahydronaphthalen-1-yl group would be a cycloalkyl group, while a 1,2,3,4-tetrahydronaphthalen-5-yl group would be an aryl group. The term “silyl” refers to a silicon moiety with three hydrocarbyl moieties attached thereto.

The term “substituted” refers to moieties having substituents replacing a hydrogen on one or more carbons or heteroatoms of the moiety. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds.

In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents, and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

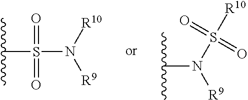

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

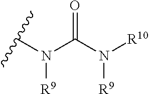

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “diagnostically effective amount” as used herein, refers to that amount of active compound or pharmaceutical agent that elicits the biological or medicinal response in a tissue system, animal or human that is being sought by a researcher, veterinarian, medical doctor or other clinician, which includes diagnosis and/or monitoring of the symptoms of the disease or disorder being treated.

The term “therapeutically effective amount” refers to that amount of a compound or pharmaceutically acceptable salt thereof which results in prevention or delay of onset or amelioration of at least one symptom of a condition disclosed herein (e.g., cancer, neurological disorder) in a subject, or an attainment of a desired biological outcome.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “unit dosage form” or “unit” as used herein refers to physically discrete units suitable as unitary dosages for human and animal subjects, each unit containing a predetermined quantity of the compound calculated in an amount sufficient to produce the desired effect in association with a pharmaceutically acceptable, diluent, carrier or vehicle. The specifications for the novel unit dosage forms of the present invention depend on the particular compound employed and the effect to be achieved, and the pharmacodynamics associated with each compound in the subject.

### Compounds

In certain embodiments, provided herein are compounds, wherein a hydroxyl in the parent compound is presented as an ester or a carbonate, an amine in the parent compound is presented as an amide, or a carboxylic acid present in the parent compound is presented as an ester. In certain embodiments, the parent compound is a neurotransmitter, an anti-depressant, an antibiotic, an antiviral, a radioactive or a fluorescent molecule. In certain embodiment the compound is a neurotransmitter or a sub-neurotransmitter specific acting ligand. In certain other embodiments, the parent compound is a ligand that targets deficient synaptic and/or neural activity, amyloid peptides or other proteins such as Tau and alpha-synuclein. In other embodiments, the invention relates to prodrug acetylated and biotinylated antisense, siRNA, miRNA and other DNA structures that target protein production.

Exemplary parent compounds include those disclosed in Tables 1 and 2. Exemplary compounds having a hydroxyl in the parent compound presented as an ester, or a carbonate or a carboxylic acid present in the parent compound presented as an ester, or an amine in the parent compound presented as an amide include those disclosed in Table 3.

Not wishing to be bound by theory, one advantage of the compounds disclosed herein is the abundance of the acetylase enzyme in the brain. The deacetylase enzyme metabolically deacetylates the compounds that cross the BBB and converts them to the parent drug with the know specific brain activity. In certain embodiments, the compound is metabolized to the active parent compound in vivo in the brain and other tissues.

TABLE 1
Exemplary Parent compounds

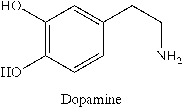 [SMILES: NCCc1ccc(O)c(O)c1]

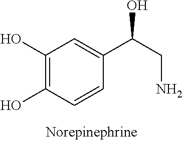 [SMILES: NC[C@H](O)c1ccc(O)c(O)c1]

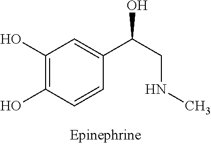

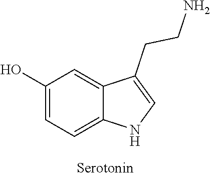 [SMILES: NCCc1c[nH]c2ccc(O)cc12]

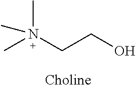

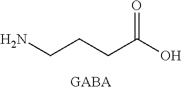

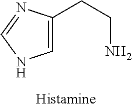

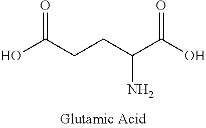

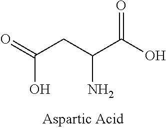

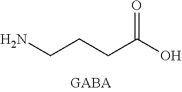

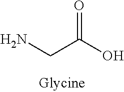

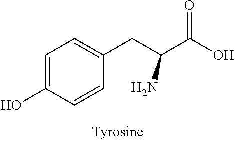 [SMILES: N[C@@H](Cc1ccc(O)cc1)C(=O)O]

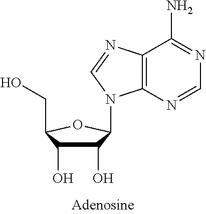

TABLE 2
Exemplary Parent Compounds

| | | |
|---|---|---|
| Tryptamine | Cromolyn | Dynorphin |
| N-methyltryptamine | Neurokinin A | Endorphin |
| Tyramine | Neurokinin B | Endomorphin |
| Octopamine | Substance P | Neuropeptide Y |
| Synephrine | Neuropeptide K | Pancreatic polypeptide |
| Phenethylamine | Somatostatin | Peptide YY |
| N-methyl- | Secretin | Vasopressin |
| phenethylamine | | |
| Adenosine | Motilin | Oxytocin |
| Adenosine | Glucagon | Neurophysin I |
| triphosphate | | |
| Serotonin (5- | Vasoactive intestinal | Neurophysin II |
| hydroxytryptamine) | peptide | |
| Dopamine | Growth hormone- | Gastrin |
| | releasing hormone | |
| Norepinephrine | Adrenocorticotropic | Cholecystokinin |
| (noradrenaline) | hormone | |
| Epinephrine | N-Acetylaspartyl- | Galanin |
| (adrenaline) | glutamate | |
| Histamine | Cocaine- and | Galanin-like peptide |
| | amphetamine- | |
| | regulated transcript | |
| Arginine | Bombesin | Anandamide |
| Asparagine | Gastrin releasing | 2-Arachidonoylglycerol |
| | peptide | |
| Glutamine | Kisspeptin | 2-Arachidonyl glyceryl |
| | | ether |
| Gamma- | Orexin A | N-Arachidonoyl |
| aminobutyric | | dopamine |
| acid | | |
| Glycine | Orexin B | Virodhamine |
| D-serine | Enkephalin | Ibuprofen |

TABLE 3

| Parent | | | |
|---|---|---|---|
| Com- | | | |
| pound | Parent Compound | | |
| Name | Structure | Exemplary Compounds | Exemplary Compounds |
| Scyllo- inositol | 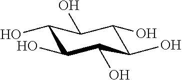 | 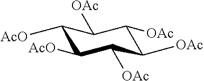 | |
| Glutamine | 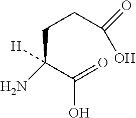 | 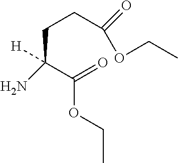 | 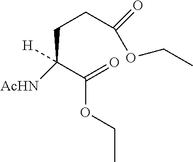 [SMILES: CCOC(=O)CC[C@H](NC(C)=O)C(=O)OCC] |
| GABA | 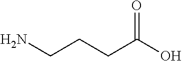 | 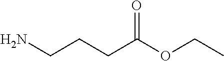 | 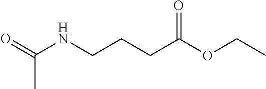 |
| Glycine | 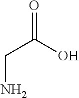 | 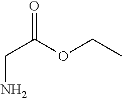 | 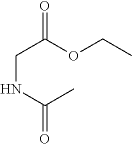 |
| Dopamine | 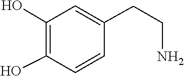 [SMILES: NCCc1ccc(O)c(O)c1] | 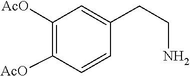 [SMILES: CC(=O)Oc1ccc(CCN)cc1OC(C)=O] | 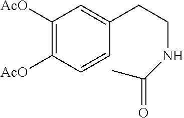 [SMILES: CC(=O)NCCc1ccc(OC(C)=O)c(OC(C)=O)c1] |
| | | 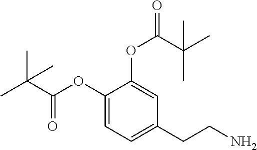 [SMILES: CC(C)(C)C(=O)Oc1ccc(CCN)cc1OC(=O)C(C)(C)C] | 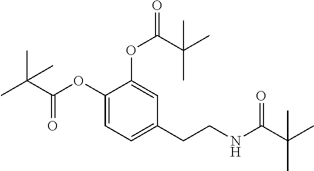 [SMILES: CC(C)(C)C(=O)NCCc1ccc(OC(=O)C(C)(C)C)c(OC(=O)C(C)(C)C)c1] |
| | | | 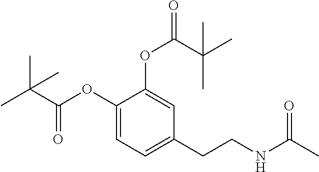 [SMILES: CC(=O)NCCc1ccc(OC(=O)C(C)(C)C)c(OC(=O)C(C)(C)C)c1] |
| 5-¹⁸F- fluoro dopamine | 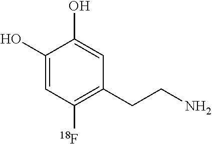 [SMILES: NCCc1cc(O)c(O)cc1[18F]] | 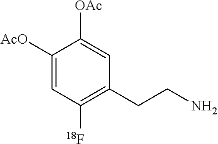 [SMILES: CC(=O)Oc1cc([18F])c(CCN)cc1OC(C)=O] | 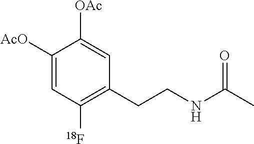 [SMILES: CC(=O)NCCc1cc(OC(C)=O)c(OC(C)=O)cc1[18F]] |
| | | 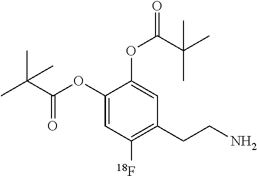 [SMILES: CC(C)(C)C(=O)Oc1cc([18F])c(CCN)cc1OC(=O)C(C)(C)C] | 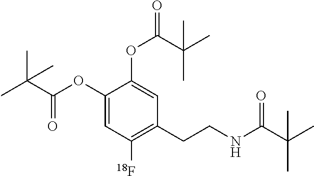 [SMILES: CC(C)(C)C(=O)NCCc1cc(OC(=O)C(C)(C)C)c(OC(=O)C(C)(C)C)cc1[18F]] |
| | | | 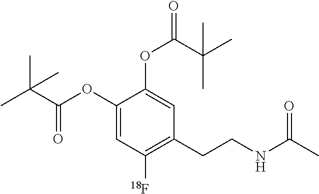 [SMILES: CC(=O)NCCc1cc(OC(=O)C(C)(C)C)c(OC(=O)C(C)(C)C)cc1[18F]] |
| Serotonin | 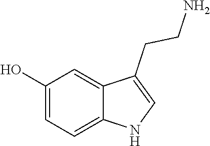 [SMILES: NCCc1c[nH]c2ccc(O)cc12] | 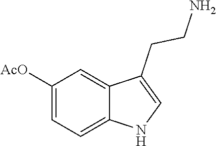 [SMILES: CC(=O)Oc1ccc2[nH]cc(CCN)c2c1] | 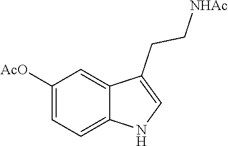 [SMILES: CC(=O)NCCc1c[nH]c2ccc(OC(C)=O)cc12] |
| L-DOPA | 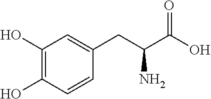 [SMILES: N[C@@H](Cc1ccc(O)c(O)c1)C(=O)O] | 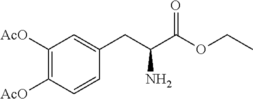 [SMILES: CCOC(=O)[C@@H](N)Cc1ccc(OC(C)=O)c(OC(C)=O)c1] | 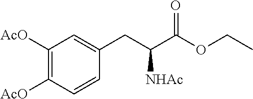 [SMILES: CCOC(=O)[C@H](Cc1ccc(OC(C)=O)c(OC(C)=O)c1)NC(C)=O] |
| Epineph- rine | 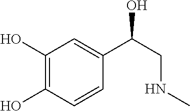 | 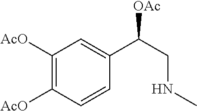 [SMILES: CNC[C@H](OC(C)=O)c1ccc(OC(C)=O)c(OC(C)=O)c1] | 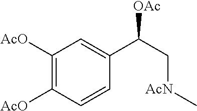 |
| Noradren- aline (Norepi- nephrine) | 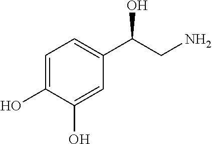 [SMILES: NC[C@H](O)c1ccc(O)c(O)c1] | 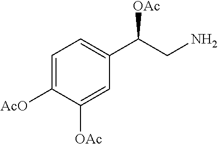 [SMILES: CC(=O)Oc1ccc([C@H](CN)OC(C)=O)cc1OC(C)=O] | 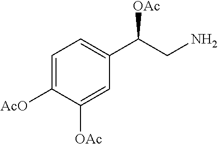 [SMILES: CC(=O)Oc1ccc([C@H](CN)OC(C)=O)cc1OC(C)=O] |
| Tyrosine | 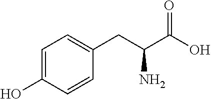 [SMILES: N[C@@H](Cc1ccc(O)cc1)C(=O)O] | 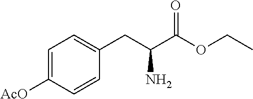 [SMILES: CCOC(=O)[C@@H](N)Cc1ccc(OC(C)=O)cc1] | 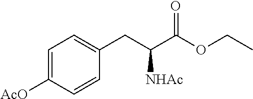 [SMILES: CCOC(=O)[C@H](Cc1ccc(OC(C)=O)cc1)NC(C)=O] |
| Aspartic acid | 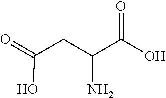 | 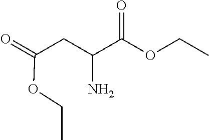 | 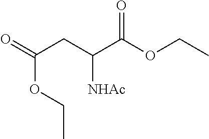 [SMILES: CCOC(=O)CC(NC(C)=O)C(=O)OCC] |
| L- Trypto- phan | 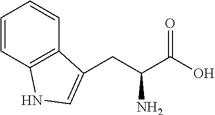 [SMILES: N[C@@H](Cc1c[nH]c2ccccc12)C(=O)O] | 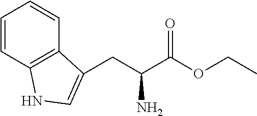 [SMILES: CCOC(=O)[C@@H](N)Cc1c[nH]c2ccccc12] | 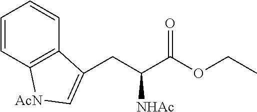 |

In certain embodiments, the compound further comprises a radioimaging agent, e.g., a radionuclide. In some embodiments, the radionuclide is selected from ³H, ¹⁸F, ³⁶Cl, ⁷⁶Br, ⁷⁷Br, ¹²³I, ¹²⁴I, ¹²⁵I, and ¹³¹I, preferably ¹⁸F. In some embodiments the compound comprises a fluorescent agent for optical imaging.

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated. The compounds herein described may have one or more charged atoms. In these embodiments, the compound may be associated with a suitable counter-ion (e.g., I⁻, Br⁻, Cl⁻, CF₃SO₃⁻). In some embodiments, the compounds may be zwitterionic, but may be neutral overall. Other embodiments may have one or more charged groups, depending on the pH and other factors. It is well known in the art how to prepare salts or exchange counter-ions. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Counter-ions may be changed, for example, by ion-exchange techniques such as ion-exchange chromatography. All zwitterions, salts and counter-ions are intended, unless the counter-ion or salt is specifically indicated. In certain embodiments, the salt or counter-ion may be pharmaceutically acceptable or may be exchanged for a pharmaceutically acceptable counter-ion, for administration to a subject. Pharmaceutically acceptable salts are discussed later.

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomer. For example, a compound of the invention may have greater than 30% de, 40% de, 50% de, 60% de, 70% de, 80% de, 90% de, or even 95% or greater de.

In certain embodiments, the present invention relates to methods of treatment with a compound disclosed herein, or a pharmaceutically acceptable salt thereof. In certain embodiments, the therapeutic preparation may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound. A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient, comprising any of the compounds shown above, and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### Pharmaceutical Compositions

In one aspect, the invention provides a pharmaceutical composition comprising a compound as disclosed herein and a pharmaceutically acceptable excipient or solvent. In certain embodiments, a pharmaceutical composition may comprise a prodrug of a compound as disclosed herein.

Embodiments of the invention include pharmaceutical compositions of the compounds disclosed herein and at least one pharmaceutically acceptable carrier or diluent. As used herein, pharmaceutical compositions include compositions suitable for administration to a subject or patient. As such, compositions do not include chemical reaction solutions or solutions used for screening assays, as these are not suitable for administration to a subject or patient. In some embodiments the compositions may include one or more than one compound of the invention, one or more other pharmaceutically active agent, and may further contain other suitable substances and excipients, including but not limited to physiologically acceptable buffering agents, stabilizers (e.g., antioxidants), flavoring agents, agents to effect the solubilization of the compound, and the like.

In other embodiments, the composition may be in any suitable form such as a solution, a suspension, an emulsion, an infusion device, or a delivery device for implantation or it may be presented as a dry powder to be reconstituted with water or another suitable vehicle before use. The composition may include suitable pharmaceutically acceptable carriers and/or excipients.

In other embodiments, the compositions may comprise an effective amount of a modulator and/or other pharmaceutically active agent in a physiologically-acceptable carrier. The carrier may take a wide variety of forms depending on the form of preparation desired for a particular route of administration. Suitable carriers and their formulation are described, for example, in Remington's Pharmaceutical Sciences by E. W. Martin.

In some embodiments, the compound may be contained in any appropriate amount in any suitable carrier substance and is generally present in an amount of 1-95% by weight of the total weight of the composition. The composition may be provided in a dosage form that is suitable for parenteral (e.g., subcutaneously, intravenously, intramuscularly, or intraperitoneally) or oral administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

In some embodiments, the compositions may be in a form suitable for administration by sterile injection. In one example, to prepare such a composition, the compositions(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). For parenteral formulations, the carrier will usually comprise sterile water, though other ingredients, for example, ingredients that aid solubility or for preservation, may be included. Injectable solutions may also be prepared in which case appropriate stabilizing agents may be employed.

Formulations suitable for parenteral administration usually comprise a sterile aqueous preparation of the compound, which may be isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery or localized delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

In some embodiments, the compositions may be in a form suitable for oral administration. In compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. If desired, tablets may be sugar coated or enteric coated by standard techniques.

Compositions suitable for oral administration may be presented as discrete units such as capsules, cachets, tablets, or lozenges, each containing a predetermined amount of the active ingredient as a powder or granules. Optionally, a suspension in an aqueous liquor or a non-aqueous liquid may be employed, such as a syrup, an elixir, an emulsion, or a draught. Formulations for oral use include tablets containing active ingredient(s) in a mixture with pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

A syrup may be made by adding the compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

In some embodiments, the composition may be in a form of nasal or other mucosal spray formulations (e.g. inhalable forms). These formulations can include purified aqueous solutions of the active compounds with preservative agents and isotonic agents. Such formulations can be adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

In some embodiments, the composition may be in a form suitable for rectal administration. These formulations may be presented as a suppository with a suitable carrier such as cocoa butter, hydrogenated fats, or hydrogenated fatty carboxylic acids.

In some embodiments, the composition may be in a form suitable for transdermal administration. These formulations may be prepared, for example, by incorporating the active compound in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, compositions of the invention may further include one or more accessory ingredient(s) selected from encapsulants, diluents, buffers, flavoring agents, binders, disintegrants, surface active agents, thickeners, lubricants, preservatives (including antioxidants), and the like.

In some embodiments, compositions may be formulated for immediate release, sustained release, delayed-onset release or any other release profile known to one skilled in the art.

In some embodiments, the pharmaceutical composition may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target the site of a pathology. For some applications, controlled release formulations obviate the need for frequent dosing to sustain activity at a medically advantageous level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, microspheres, molecular complexes, nanoparticles, patches, and liposomes.

In some embodiments, the composition may comprise a “vectorized” form, such as by encapsulation of the compound in a liposome or other encapsulate medium, or by fixation of the compound, e.g., by covalent bonding, chelation, or associative coordination, on a suitable biomolecule, such as those selected from proteins, lipoproteins, glycoproteins, and polysaccharides.

In some embodiments, the composition can be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents. Alternatively, the compound may be incorporated in biocompatible carriers, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly (lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

In all embodiments, the compound or other active compounds may be present as pharmaceutically acceptable salts or other derivatives. Derivatives include all individual enantiomers, diastereomers, racemates, and other isomers of the compounds. Derivatives also include all polymorphs and solvates, such as hydrates and those formed with organic solvents, of the compounds. Such isomers, polymorphs, and solvates may be prepared by methods known in the art, such as by regiospecific and/or enantioselective synthesis and resolution.

The ability to prepare salts depends on the acidity or basicity of the compounds. Suitable salts of the compounds include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, α-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the excipient is one that temporarily disrupts the BBB (e.g., mannitol). Such an excipient could be part of any formulation disclosed herein for oral, nasal, powder, injectable, IV, IP, IM or other routes of administration. The excipients are added in an appropriate amount to control brain uptake delivery. Unless the context clearly indicates otherwise, compositions of all embodiments can comprise various pharmaceutically acceptable salts, or other derivatives described above.

The formulation and preparation of such compositions are well known to those skilled in the art of pharmaceutical formulation. Formulations can be found in Remington: The Science and Practice of Pharmacy.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

Dosing can be single dosage or cumulative (serial dosing), and can be readily determined by one skilled in the art. For instance, treatment may comprise a one-time administration of an effective dose of a pharmaceutical composition disclosed herein. Alternatively, treatment may comprise multiple administrations of an effective dose of a pharmaceutical composition carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, the period of administration of a therapeutic compound is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In certain embodiments, a treatment regimen may comprise a period during which administration is stopped for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The patient receiving this treatment is any animal in need, including primates, in particular humans, and other mammals such as equines, cattle, swine and sheep; and poultry and pets in general.

### Methods

### Neurological Disease

In certain aspects, the invention provides methods for treating or preventing a neurological disease by administering a compound or composition disclosed herein. Exemplary forms of neurological and mental diseases that may be treated by the methods that include, but are not limited to, schizophrenia, depression, anxiety, attention deficit or hyper activity disorders, personality disorders, schizotypal personality disorder, avoidant personality disorder, social phobia, histrionic personality disorder, and somatization disorder, drug addiction, obesity, Alzheimer's disease, neurofibromatosis, Huntington's disease, amyotrophic lateral sclerosis, multiple sclerosis, stroke, prion, Parkinson's disease, dystonias, and dementia.

When administering a compound to an organism, the compound may be administered by any suitable means. Examples of routes of administration include oral administration, rectal administration, topical administration, inhalation (nasal) or injection. Administration by injection includes intravenous (IV), intramuscular (IM), and subcutaneous (SC) administration. The pharmaceutical compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, and intrabronchial.

In one embodiment, the invention provides a method wherein the subject is a human, rat, mouse, cat, dog, horse, sheep, cow, monkey, avian, or amphibian. In another embodiment, the cell is in vivo or in vitro. Typical subjects to which compounds of the invention may be administered will be mammals, particularly primates, especially humans. For veterinary applications, a wide variety of subjects will be suitable, e. g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

### Cancer

In certain aspects, the invention provides methods for treating or preventing cancer (e.g., cancer of the brain) by administering a compound or composition disclosed herein. The actual symptoms associated with cancer are well known and can be determined by a person of ordinary skill in the art by considering one or more factors, including, without limitation, the location of the cancer, the cause of the cancer, the severity of the cancer, and/or the tissue or organ affected by the cancer. Those of skill in the art will know the appropriate symptoms or indicators associated with a specific type of cancer and will know how to determine if an individual is a candidate for treatment as disclosed herein. Exemplary forms of cancer include but are not limited to glioblastoma, astrocytoma, glioma, primary CNS lymphoma, and fibrillary astrocytoma.

### Radioimaging

The radioimaging agents of the invention may be used in accordance with the methods of the invention by those of skill in the art, e.g., by specialists in nuclear medicine, to image tissue in a mammal. Any mammalian tumor may be imaged the imaging agents of the invention. Images are generated by virtue of differences in the spatial distribution of the imaging agents which accumulate in the various tissues and organs of the mammal. The spatial distribution of the imaging agent accumulated in a mammal, in an organ, or in a tissue may be measured using any suitable means, for example, a PET or single photon emission computer tomography (SPECT) imaging camera apparatus, and the like.

PET imaging is accomplished with the aid of tracer compounds labeled with a positron-emitting isotope (Goodman, M. M. Clinical Positron Emission Tomography, Mosby Yearbook, 1992, K. F. Hubner et al., Chapter 14). These tracer compounds can be labeled with a positron-emitting radionuclide that includes ¹⁸F and ⁷⁶Br. In general, a PET label, is a label which is covalently attached to the remainder of a molecule and should have a half-life of at least about 5-20 minutes, preferably about 60 minutes or more. Examples of PET labels include ¹⁸F, ¹³N, ⁷⁶Br ⁷⁷Br, ⁶²Cu, ⁶⁴Cu, and ⁸²Rb.

For SPECT imaging, the inventive compound can be labeled with a γ-emitting nuclide, such as ^{99m}Tc, ¹¹¹In, ⁶⁷Ga, ¹²³I, ¹³¹I, and others.

### EXEMPLIFICATION

The invention now being generally described, it will be more readily understood by reference to the following examples, which are included merely for purposes of illustration of certain aspects and embodiments of the present invention, and are not intended to limit the invention.

### Example 1—Fluorinated Inositol

Myo-inositol or its hexaphosphate derivative, ingested or made endogenously from glucose, are metabolized by CDP-diacylglycerol to form phosphatidylinositol, a component of cell membranes. Phosphatidylinostiol is cleaved by phospholipase C to form diacylglycerol (DAG), and after subsequent enzymatic activity, a variety of mono-, di-, tri- and tetraphosphate inositols. Two of these inositol derivatives produced are second-messenger molecules that control cellular processes such as cell growth, transformation, and neuronal signaling. Inositol-1,4,5-triphosphate modifies intracellular calcium levels and inositol-3,4,5-triphosphate is involved in signal transduction.

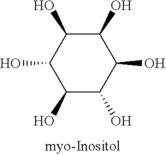 [SMILES: O[C@H]1[C@H](O)[C@@H](O)[C@H](O)[C@@H](O)[C@H]1O]

Previously, inositol hexaphosphate (IP6), found in large amounts in cereals, was proposed as a promising anti cancer agent because of its effect on several cancers in blood, colon, liver, lung, mammary and prostate. IP6 reduced the rate of cellular proliferation, both in vivo and in vitro, and has exhibited an ability to reduce DNA synthesis. In vitro studies showed that malignant cells rapidly accumulated IP6 intracellularly. Moreover, a decreased rate of cell proliferation occurred and malignant cells reverted to a non-cancerous state. This phenomenon has been observed in HT-29 human colon carcinoma cells, HepG2 cells, fibrosarcoma cells, MCF7 cells, and ovarian cancer cells. In one example, rats fed sodium inositol hexaphosphate prior to carcinogen treatment exhibited a 35% decrease in large intestinal cancer compared to the control carcinogen group. It has been suggested that IP6 exerts cellular control by interfering with mineral absorption.

In vitro studies focusing on the uptake and metabolism of tritiated myo-inositol by FI210 murine leukemia cells showed a rapid uptake rate that was directly proportional to the extracellular concentration. Incorporation of the tracer continued in a linear fashion over a 4-hour period. Similar accumulation of [2-³H]-myo-inositol in C6 glioma cells is known to occur.

Described herein, fluorine-18 labeled 2-deoxy-2-fluoro-myo-inositol and 4-deoxy-4-fluoro-myo-inositol are regioselectively prepared as potential probes for PET. Briefly, myo-inositol (0.06 mol), triethyl orthoformate (0.09 mol), p-toluenesulfonic acid monohydrate (1.0 g) and dry dimethylformamide (100 mL) were heated at 100° C. for 3 hrs. After cooling, triethylamine (4 mL) was added, volatiles were removed in vacuo, and chromatography (silica gel, 20% methanol/methylene chloride) afforded the 1,3,5-orthoformate in 70% yield. Myo-inositol 1,3,5-orthoformate was treated with 1 eq. of methanesufonyl chloride in pyridine which resulted in sulfonylation of the 2-hydroxyl group. 2-O-mesyl-myo-inositol 1,3,5-orthoformate was dibenzoylated and then the orthoformate moiety was removed using methanesulfonic acid in methanol. The product was then benzoylated to give 1,3,4,5,6-O-pentabenzoyl-2-O-mesyl-myo-inositol. The 4-O-mesyl isomer was prepared from 2,6-O-dibenzoyl-myo-inositol 1,3,5-orthoformate and methanesulfonyl chloride in pyridine (11). The orthoformate moiety was removed and the crude material was benzoylated to give 1,2,3,5,6-O-pentabenzoyl-4-O-mesyl-myo-inositol. Radiofluorination of the mesylates was performed in a sealed vial containing dry K¹⁸F/Kryptofix in acetonitrile at 150° C. for 10 min. The reaction mixture was passed through a silica gel Sep-Pak using methylene chloride (3 mL) and solvent removed. The crude material was treated with 1 mL of a solution of 2M ammonia in methanol at 100° C. for 15 min, solvent removed, and products were purified on alumina and C₁₋₈ Sep-Paks (in series) using saline.

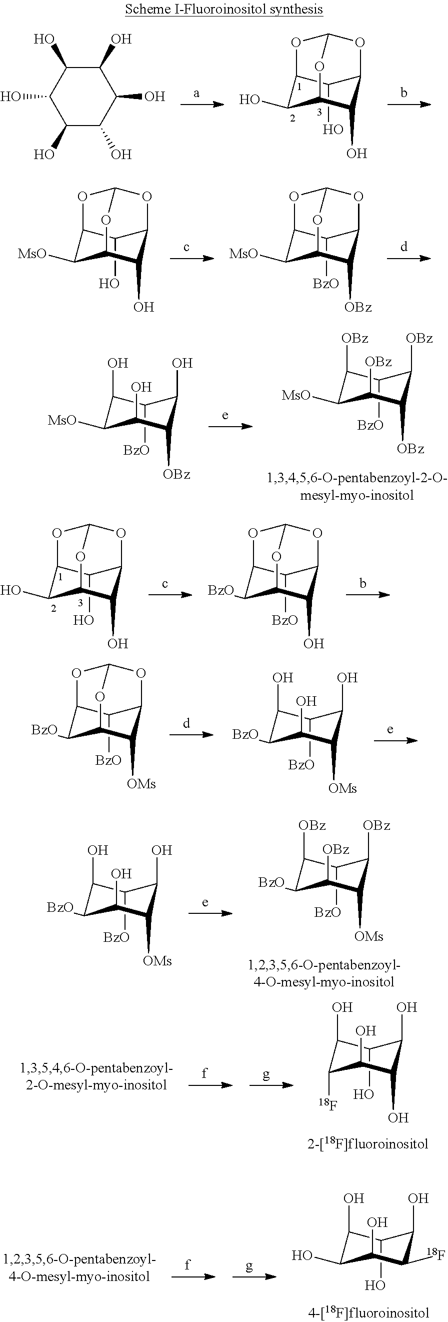

Reagents and conditions for the reaction sequence in Scheme I: a) triethyl orthoformate (0.09 mol), p-toluenesulfonic acid monohydrate, DMF, 100° C., 3 hr; b) 1 equiv. methanesufonyl chloride, pyridine, 0° C. 2 hr, 25° C., 16 hr; c) benzoyl chloride 2 equiv., pyridine, 25° C., 16 hr; d) p-toluene-sulfonic acid monohydrate, methanol, 40° C., 2 hr; e) benzoyl chloride 3.3 equiv., pyridine, 25° C.; f) K¹⁸F/K222 complex dried with acetonitrile, 2-O-mesyl-myo-inositol isomer or 4-O-mesyl-myo-inositol isomer, 120° C., 10 min; g) 2M ammonia in methanol, 100° C., 15 min.

Radiofluorination yields were 10% for 2-[¹⁸F]-fluoroinositol and 30-40% for 4-[¹⁸F]-fluoroinositol.

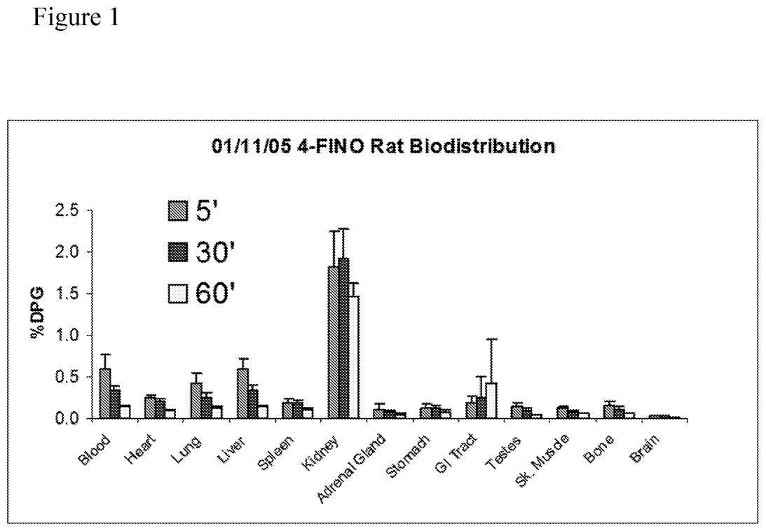

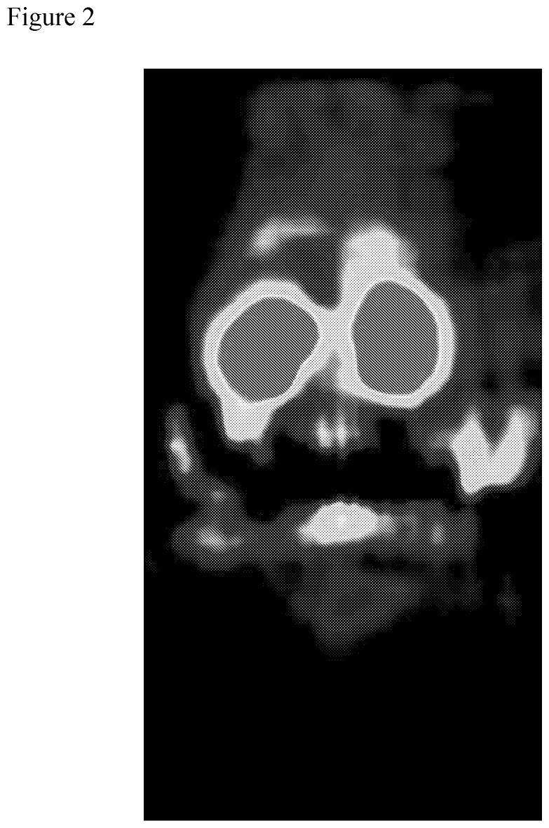

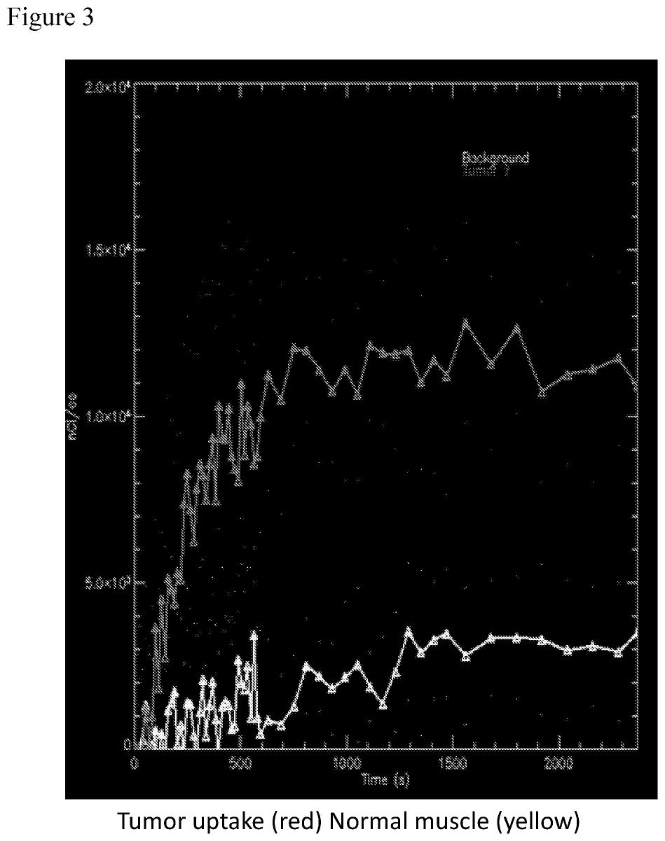

The tumor localizing ability of 4-[¹⁸F]-fluoroinositol was tested in a mouse bearing a small prostate tumor (PC3) (<100 mg). Biodistribution of 4-[¹⁸F]-fluoroinositol in normal rats showed high and fast accumulation in kidney and bladder without any other specific tissue accumulation, and defluorination was minimal (FIG. 1). Using 4-[¹⁸F]-fluoroinositol, MircoPET imaging of an invisible prostate tumor expression (<100 mg) in mice implanted in the thigh showed positive accumulation in the tumor area (FIGS. 2 and 3).

This, a fluorine-18 labeled inositol may be used in early cancer detection using PET imaging.

### Example 2—Comparison of Scyllo-Inositol and Acetylated Scyllo-Inositol

Scyllo-inositol has been shown to directly interact with amyloid beta oligomers inhibiting Aβ42 fiber formation in the brain. Mechanistic data indicate scyllo-inositol binds and neutralizes these oligomers into soluble complexes while reducing the amount of larger oligomeric species. Currently, scyllo-inositol is being investigated as a treatment for Alzheimer's disease. Recently, the analog 1-deoxy-1-fluoro-scyllo-inositol also was reported to significantly inhibit the formation of Aβ42 fibers. Hence, this ¹⁸F-labeled scyllo-inositol derivative may be beneficial as a probe for studying the early formation of amyloid plaque. Disclosed herein is 1-deoxy-1-[¹⁸F]-fluoro-2,3,4,5,6-penta-O-acetyl-scyllo-inositol (1) and 1-deoxy-1-[¹⁸F]-fluoro-scyllo-inositol (2), and their biodistributions.

### Synthesis of 1 and 2

1,2-O-Cyclohexylidene myo-inositol 3. Myo-Inositol (5 g, 28 mmol) cyclohexanone (50 mL), p-toluene sulfonic acid (36 mg), DMF (5 mL), and benzene (25 ml) were refluxed in a Dean-Stark apparatus for 16 hr. The clear solution was cooled to 40° C. and benzene (25 ml), petroleum ether (25 ml), and ethanol (12 mL) were added. To this solution was added p-toluene sulfonic acid (0.3 g) and the mixture was stirred at 4° C. for 2 hr. Triethylamine (0.3 ml) was added and the mixture was allowed to stand at −20° C. for 16 hr. The suspension was filtered and the filtrate was heated in ethanol (100 ml) and triethylamine (0.5 ml) at 80° C. for 1 hr. After cooling, cis-1,2-O-cyclohexylidene myo-inositol 3 was collected by filtration; 5.1 g (70%); m.p. 176-177° C.

1,4,5,6-Tetra-O-benzyl-2,3-O-cyclohexylidene 4. Compound 3 (14.4 g, 55.2 mmol) was treated with benzyl chloride (139.8 g, 1.11 mol) and sodium hydride (20.9 g, 828 mmol) in DMF (150 ml) and the mixture was heated at 100° C. for 16 hr. The solution was poured over ice-water and extracted with ether. The combined extracts were washed with water (50 ml), brine (50 ml) and dried. Solvent was evaporated in vacuo and the residue was chromatographed with a mixture of toluene:butanone (20:1) on silica gel to give 4; 19.5 g (57%); m.p. 83-85° C.

3,4,5,6-Tetra-O-benzyl-myo-inositol 5. Compound 4 (19.5 g, 31.4 mmol) was heated for 4 hr at 100° C. with glacial acetic acid (150 ml) and water (35 ml). The solution was evaporated in vacuo and the residue was chromatographed on silica gel to give 5, 10.8 g (64%); mp 124-126° C.

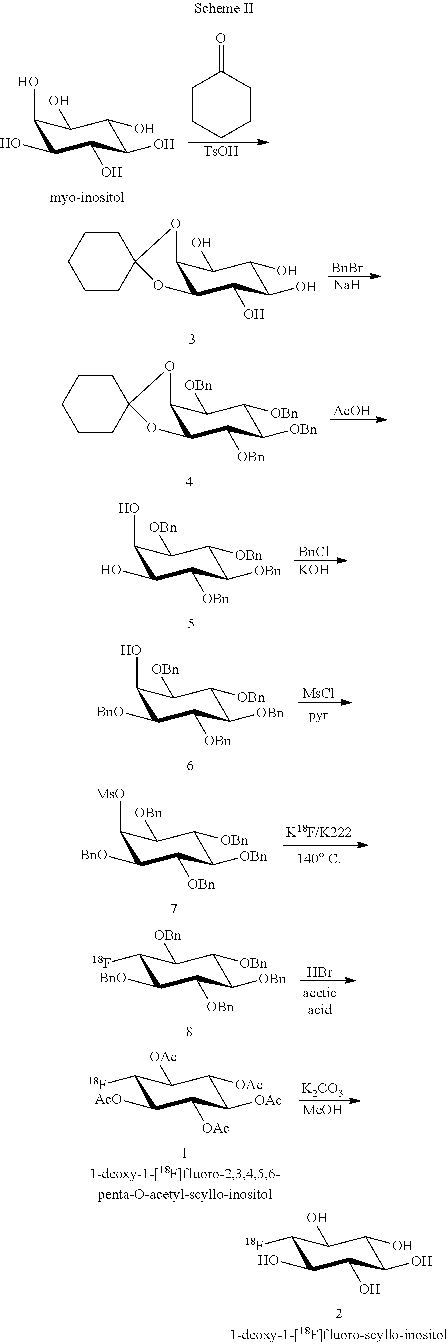

2,3,4,5,6-Penta-O-benzyl-myo-inositol 6. A solution of 5 (10.8 g, 20 mmol) in benzene (50 ml) was treated with benzyl chloride (111 g, 880 mmol) and KOH (3 g, 95%, 120 mmol). The mixture was stirred at 100° C. for 2 hr. The reaction mixture was poured over ice-water (70 ml) and extracted with ether (200 ml). The organic layer was separated and washed successively with brine (50 ml). The solution was evaporated in vacuo and the residue was chromatographed with EtOAc/Hexane on silica gel to give 6; 7.1 g (56%); mp 124-125° C. [lit.(5), 125-127° C.], 1,3,4,5,6-Penta-O-benzyl-2-O-methanesulfonyl myo-inositol 1. Compound 6 (1 g, 2.6 mmol) in anhydrous pyridine (5 mL) and methylene chloride (20 mL) was treated with DMAP (2 mg) and methanesulfonyl chloride (0.6 g, 5.2 mmol) at 0° C. for 15 min and then stirred at 25° C. for 16 hr. The solution was evaporated in vacuo and the residue was chromatographed on silica gel to give 7; ¹H NMR (300 MHz, CDCl₃): 3.0 (3H, s, CH₃), 3.48 (1H, t, 5-H), 3.5 (2H, dd, 1-H, 3-H), 3.88 (2H, t, 4-H, 6-H), 4.6-4.9 (10H, m, CH₂), 5.35 (1H, t, 2-H), 7.33 (25H, m, aromatic). 1-Deoxy-1-[¹⁸F]-fluoro-2,3,4,5,6-penta-O-acetyl-scyllo-inosilol 1. A Wheaton 5-mL reaction vial containing fluorine-18 (100 mCi) in 1 mL O-18-enriched water, Kryptofix (8 mg), and potassium carbonate (2 mg) was heated at 120° C. and water was evaporated with the aid of a nitrogen gas flow. The K¹⁸F/Kryptofix complex was dried three successive times by the addition of 1 mL acetonitrile followed by evaporation of the solvent using a nitrogen flow. A solution of 2 mg of mesylate 7 in 0.1 mL acetonitrile was added to the sealed vial and fluorination was performed at 140° C. for 10 min. Once cooled to room temperature, the reaction mixture was passed through a silica gel SepPak using methylene chloride (3 mL) and solvent was removed using a nitrogen flow. A 33% wt. solution of HBr in acetic acid (0.2 mL) was added to the vial at 25° C. After 15 min, solvent was removed by a nitrogen stream. The crude product was purified on a silica gel SepPak using 10% methanol in methylene chloride. Solvent was removed and 1 was dissolved in 10% ethanol/saline and filtered (MillexGV 0.22 mm).

Deoxy-1-[¹⁸F]-fluoro-scyllo-inositol 2 Compound 7 was dissolved in 0.5 mL of 10% aq. K₂CO₃ in methanol and the mixture was heated at 80° C. for 20 min. Solvent was reduced and 1-deoxy-1-[¹⁸F]-fluoro-scyllo-inositol was purified on a C18 Sep-Pak using saline and filtered (MillexGV 0.22 mm).

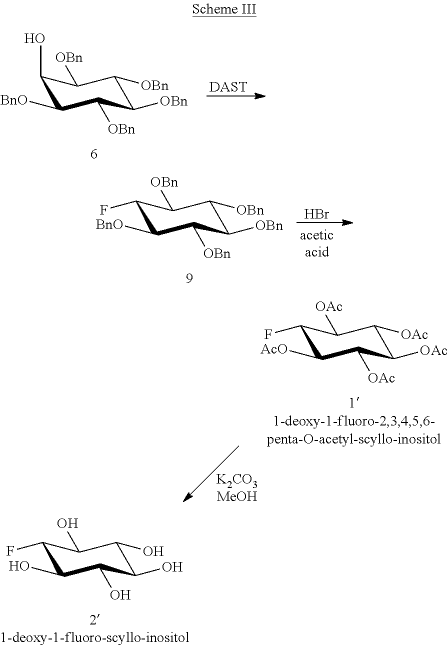

Deoxy-1-fluoro-2,3,4,5,6-penta-O-acetyl-scyllo-inositol V A solution of 6 (0.4 g, 0.064 mmol) in dry THF (20 mL) and triethylamine (1 mL) was cooled to 0° C. before addition of DAST (140 μL, 1.1 mmol) under N2. The reaction mixture was stirred for at 25° C. for 16 h. The mixture was added to cold sat'd NaHCCl₃ and washed with brine. Chromatography on silica gel using methylene chloride/hexane (97:3) give 9 (140 mg, 35%). A 33% wt. solution of HBr in acetic acid (1 mL) was added to a vial containing 9 (100 mg) at 25° C. and the mixture was stirred for 4 h. Solvent was removed in vacuo and chromatography on silica gel using ethyl acetate/hexane (5:95) give V (42 mg, 70%); mp 242-244° C.; 1H NMR (300 MHz, CDCl₃): 1.99 (3H, s, CH₃), 2.00 (3H, s, CH₃), 2.01 (3H, CH₃), 2.08 (3H, s, CH₃), 2.09 (3H, s, CH₃), 4.55 (1H).

Radiofluorination yields were 20% and 10% for ¹⁸F-scyllo-inositols 1 and 2, respectively.

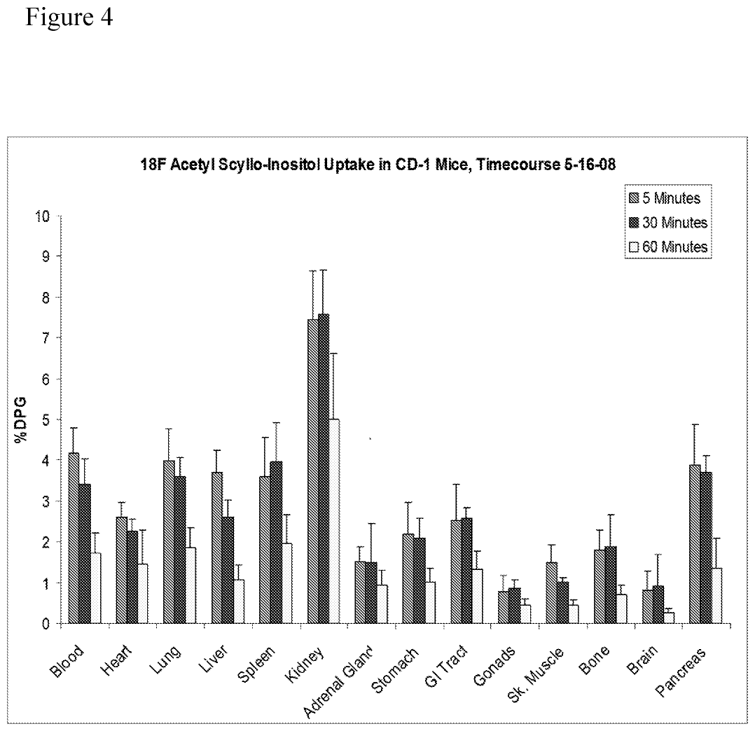

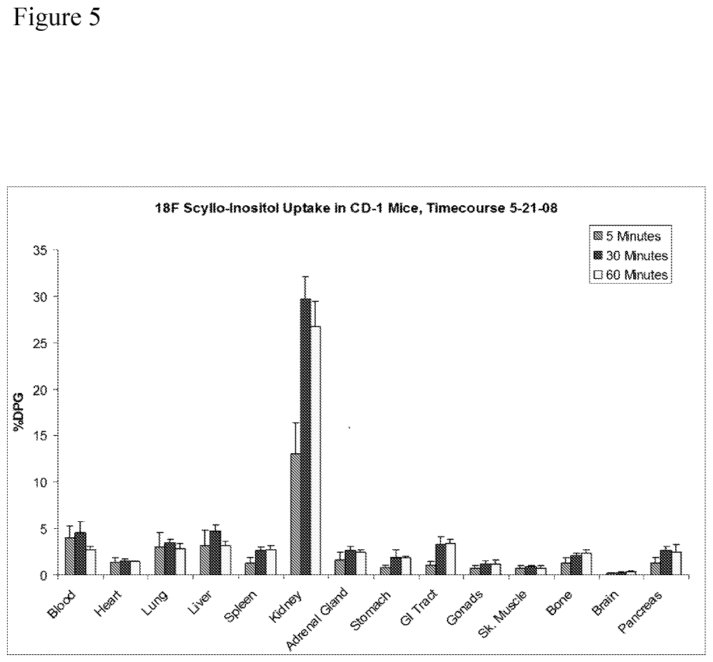

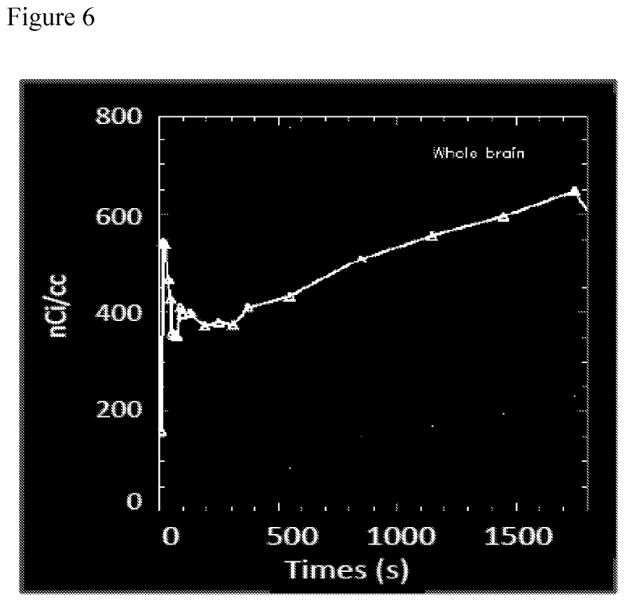

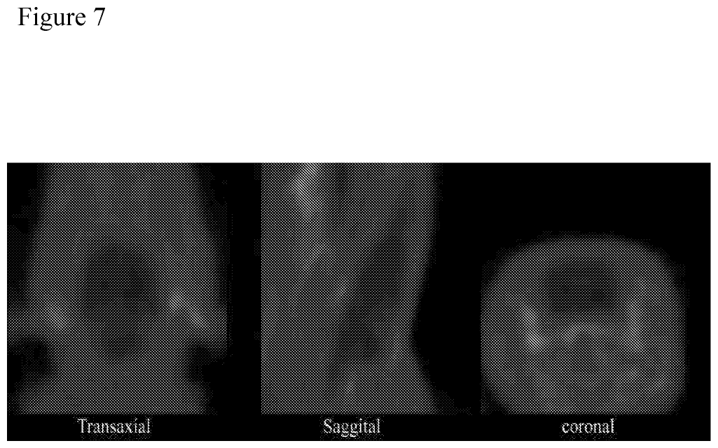

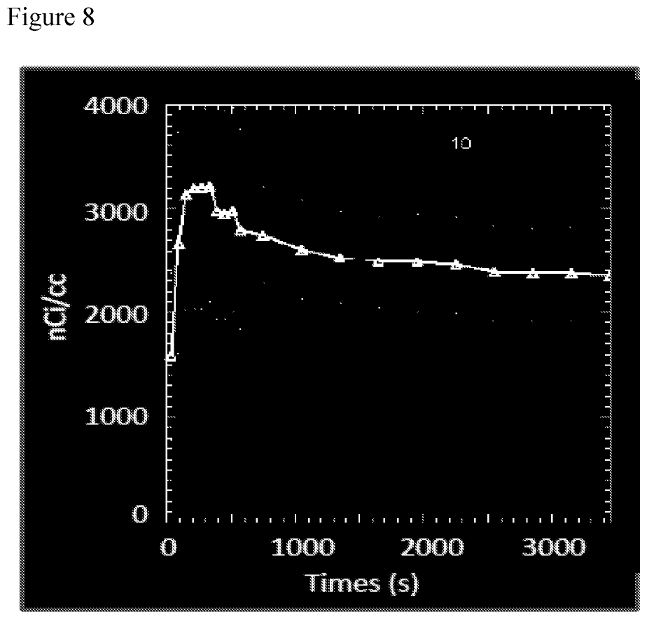

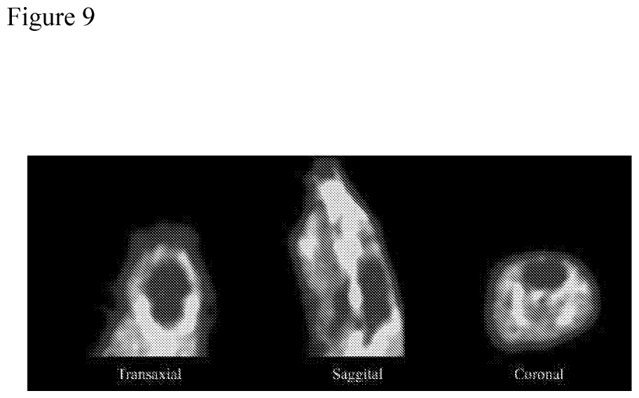

Brain uptake (% DPG) at 5, 30, and 60 min for the acetylated derivative 1 was 0.8%, 0.9%, and 0.26% (See FIG. 4). Brain uptake for derivative 2 was 0.18%, 0.30%, and 0.36%, with the majority of the activity accumulating in kidneys (See FIG. 5).

Inositol is transported across the blood-brain barrier by a low capacity, saturable system. Here, the results show that the brain uptake at 30 min for the penta-acetylated derivative 1 was three times higher than that for the penta-hydroxyl derivative 2. The higher uptake for the penta-acylated compound may be due, in part, to the increased lipophilicity [predicted by a Log P], Compound 1 also showed significant brain clearance in normal rats at 60 min.

c Log P: scyllo-inositol (−2.59); isomer 1 (1.37); isomer 2 (−1.48).

Acetylated [¹⁸F]-fluoro-scyllo-inositol 1 exhibited three times higher brain accumulation within 30 min than that of [¹⁸F]fluoro-scyllo-inositol 2. Compound 1 also showed significant brain clearance in normal rats at 60 min. These results suggest that studies examining binding of the acetylated compounds to to soluble oligomers and to Alzheimer brain tissue are warranted. Formulation of the acetylated compounds for dermal uptake can have a beneficial effect on the compliance of drug treatment in neurodegeneration.

The parameters of compounds affecting their ability to penetrate the blood-brain penetration are shown in Table 4.

TABLE 4
Suggested Physicochemical Property Ranges for
Increasing the Potential for BBB Penetration.

| | top 25 | | % of top 25 | | % of top 25 |
|---|---|---|---|---|---|
| | CNS | | CNS drugs | | CNS drugs |
| | drugs | suggested | in suggested | preferred | in preferred |
| Property | mean | limits | range | range | range |
| PSA | 47 | <90 | 96 | <70 | 76 |
| (A²) | | | | | |
| cLogP | 2.8 | 2-5 | 68 | 2-4 | 52 |
| clogD | 2.1 | 2-5 | 61 | 2-4 | 61 |
| (pH 7.4) | | | | | |
| MW | 293 | <500 | 100 | <450 | 100 |

### Example 3—1-Deoxy-1-[¹⁸F]-Fluoro-D-Mannitol to Facilitate BBB Permeability Evaluation

High molecular weights of peptides and proteins, as well as their Log P, and PSA values render these molecules poorly suitable for the BBB penetration. However, in certain instances of brain disorders or cancer, there is a BBB disruption that allows leakage and BBB diffusion. Other potential transport mechanism is via facilitated diffusion and/or active transport.

Another mechanism for large molecule transport is BBB disruption with agents like mannitol.

In the present disclosure mannitol is radiofluorinated by exchanging an OH on position 1 for ¹⁸F. Rat brain imaging, post cold mannitol infusion doubled brain concentration of the radiolabeled agent, indicating the increased uptake of added non-labeled mannitol infusion.

Brain imaging of subjects following mannitol infusion and pre chemotherapy treatment may improve prognostic outcome of the chemotherapy treatment.

Evaluation of 1-deoxy-1-[¹⁸F]-fluoro-D-mannitol in rat brain as a potential tracer for evaluating BBB permeability prior to chemotherapy administration is disclosed herein. Additionally, application of 1-deoxy-1-[¹⁸F]-fluoro-D-mannitol to monitoring brain permeability changes during intracranial chemotherapy administration is disclosed. Here, imaging is performed in normal rats and rats pretreated with mannitol.

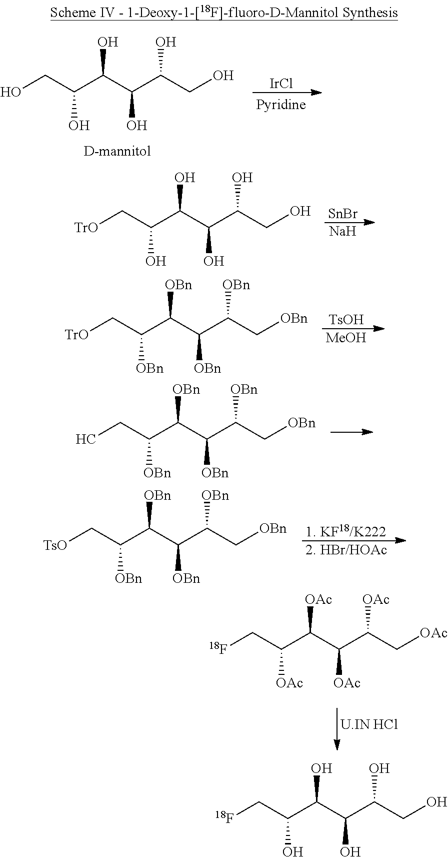

1-deoxy-1-[¹⁸F]-fluoro-D-mannitol. A Wheaton 5-mL reaction vial containing fluorine-18 (100 mCi) in 1.4 mL O-18-enriched water, Kryptofix (8 mg), and potassium carbonate (2 mg) was heated at 120° C. and water was evaporated with the aid of a nitrogen gas flow. The K¹⁸F/Kryptofix complex was dried three successive times by the addition of 1 mL acetonitrile followed by evaporation of the solvent using a nitrogen flow. A solution of 2 mg of tosylate in 0.5 mL acetonitrile was added to the sealed vial and fluorination was performed at 120° C. for 10 min. Once cooled to room temperature, the reaction mixture was passed through a silica gel SepPak using 10% methanol in methylene chloride (3 mL) and solvent was removed using a nitrogen flow. A 33% wt. solution of HBr in acetic acid (0.2 mL) was added to the vial at 25° C. After 15 min, solvent was removed by a nitrogen stream and the crude pentaacetate product was hydrolyzed with 1 mL of 0.1M HCl at 130° C. for 15 min. The 1-[¹⁸F]-fluoro-D-mannitol was purified with a series of columns: Ag11×8 ion-retarding resin (3 g BioRad), alumina Sep-Pak (Waters) and C18-Pak (Waters) using water as the eluent. The time required for synthesis and purification was 1.5 h the radiochemical yield was 20-30% (end of synthesis, EOS). Radiochemical purity was >98% based on radio-TLC (acetonitrile/water 5:95).

Imaging Studies Using 1-Deoxy-1-[¹⁸F]-Fluoro-D-Mannitol.

PET imaging was performed in a rats using a Siemens Focus 220 scanner with Neurological CT scanner. [¹⁸F]-fluoromannitol (1.2 mCi) was injected via tail-vein and imaged in list mode for one hour.

In a separate experiment, a 25% solution of D-mannitol was i.v. injected 30 min prior to tracer injection.

The tracer was produced in 1.2 hours with yields of 10-20% (EOS) and >98% radiochemical purity. PET imaging showed very fast flow related brain accumulation and washout in the first 60 sec. Brain accumulation increased to 0.1% injected dose (% ID) at 47 second and decreased to 0.04% ID at 5 min. Surprisingly, brain activity increased and doubled to 0.08% ID at 1 hour (standard uptake value (SUV) at 1 hour was 0.3). The images clearly indicated high brain uptake of the agent. The increased brain uptake is associated with [¹⁸F]-Fluoromannitol release from blood plasma and other tissues.

### Example 4-6-[¹⁸F]-Fluorodopamine Synthesis and Imaging Studies

6-[¹⁸F]-fluorodopamine and its derivatives were synthesized according to procedures adapted from J. Zischler, N. Kolks, D. Modemann, B. Neumaier, B. D. Zlatopolskiy “Alcohol-Enhanced Cu-Mediated Radiofluorination,” Chemistry. A European Journal, 23(14):3251-3256 (2017).

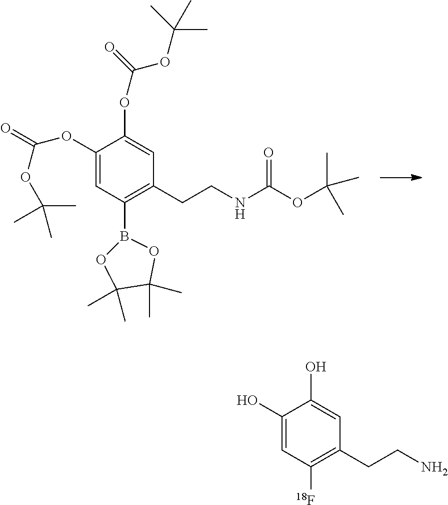 [SMILES: CC(C)(C)OC(=O)NCCc1cc(OC(=O)OC(C)(C)C)c(OC(=O)OC(C)(C)C)cc1B1OC(C)(C)C(C)(C)O1 ; NCCc1cc(O)c(O)cc1[18F]]

6-[¹⁸F]fluorodopamine: K¹⁸F (400 mCi) in water was loaded onto a QMA-carb (Waters) cartridge and the cartridge was dried using acetone (6 mL) and a nitrogen stream for 10 min. ¹⁸F was eluted from the cartridge with a solution of Et₄NHCO₃ (5 mg) in n-BuOH (400 μL) into a 5-mL reaction vial. A solution of the N,O,O-tri-Boc-6-(4,4,5,5-tetramethyl-1,3,2-dioxaborolan-2-yl)dopamine (60 μmol) and Cu(OTf)₂(py)₄ (36 mg, 53 μmol) in DMA (800 μL) was added.

The reaction mixture was heated at 110° C. for 10 min under air. Afterwards, the reaction mixture was quenched with water (80 mL) and passed through a C₁₋₈ cartridge (500 mg), preconditioned with ethanol (30 mL). The cartridge was washed with water (30 mL). The protected product was eluted with acetonitrile (2 mL), and the resulting solution was concentrated to dryness at 80° C. Then 12 M HCl (300 μL) was added to the residue, and the reaction mixture was stirred at 130° C. for 10 min. Acetonitrile (2 mL) was added, and the resulting solution was concentrated to dryness at 80° C. The crude radiolabeled product was dissolved in 4% EtOH in 0.02 M NaH₂PO₄ (500 μL, pH 2.5) and purified by HPLC to afford the desired tracer in a solution ready for injection. HPLC conditions for purification: Phenomenex luna C-18 250×10 mm; eluent: 0.02 M NaH₂PO₄, pH 3.6); flow rate: 7 mL/min; t_{R}=22 min, 6-[¹⁸F]-fluorodopamine; yield was 20% end of bombardment (EOB).

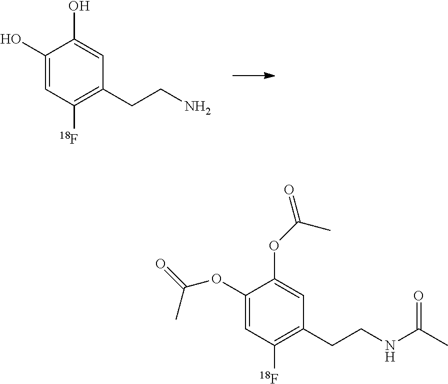 [SMILES: NCCc1cc(O)c(O)cc1[18F] ; CC(=O)NCCc1cc(OC(C)=O)c(OC(C)=O)cc1[18F]]

[¹⁸F]-N-[2-[3,4-Bis(acetyloxy)-6-fluorophenyl]ethyl] acetamide (F-18-6FPBA): Crude 6-[¹⁸F]-fluorodopamine was diluted with 2 mL of saturated sodium bicarbonate solution, and acetic anhydride (400 μL) was added to the stirred solution followed by addition of solid sodium bicarbonate (0.2 g). The mixture was stirred for 15 min at 25° C. The crude radiolabeled product mixed with acetonitrile (2 mL) and purified by HPLC (Phenomenex luna C-18, C-18 250×10 mm, eluent: 40/60 acetonitrile/water); flow rate: 5 mL/min).

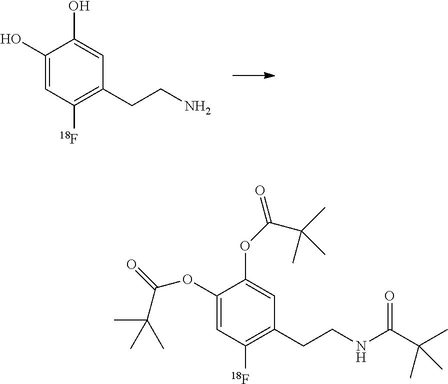 [SMILES: NCCc1cc(O)c(O)cc1[18F] ; CC(C)(C)C(=O)NCCc1cc(OC(=O)C(C)(C)C)c(OC(=O)C(C)(C)C)cc1[18F]]

[¹⁸F]-2,2-Dimethyl-4-[2-[(2,2-dimethyl-1-oxopropyl)amino] ethyl]-1,2-6-fluorophenylene propanate ester (F-18-6FPBPF): Crude 6-[¹⁸F]-fluorodopamine was diluted with 2 mL of saturated sodium bicarbonate solution, and trimehtylacetyl chloride (400 uL) was added to the stirred solution followed by addition of solid sodium carbonate (0.2 g). The mixture was stirred for 15 min at 25° C. The crude radiolabeled product was mixed with acetonitrile (2 mL) and purified by HPLC (Phenomenex luna C-18, C-18 250×10 mm, eluent: 50/50 acetonitrile/water); flow rate: 5 mL/min).

Imaging Studies Using 6-[¹⁸F]-Fluorodopamine and Derivatives

Dynamic PET scans (microPET/CT, Trifoil camera) were acquired for 60 min after a bolus injection (200-300 μCi) of 6-[¹⁸F]-fluorodopamine and 6-[¹⁸F]-fluorodopamine analogs into the tail vein of rats (140-160 g) under anesthesia (2% isoflurane).

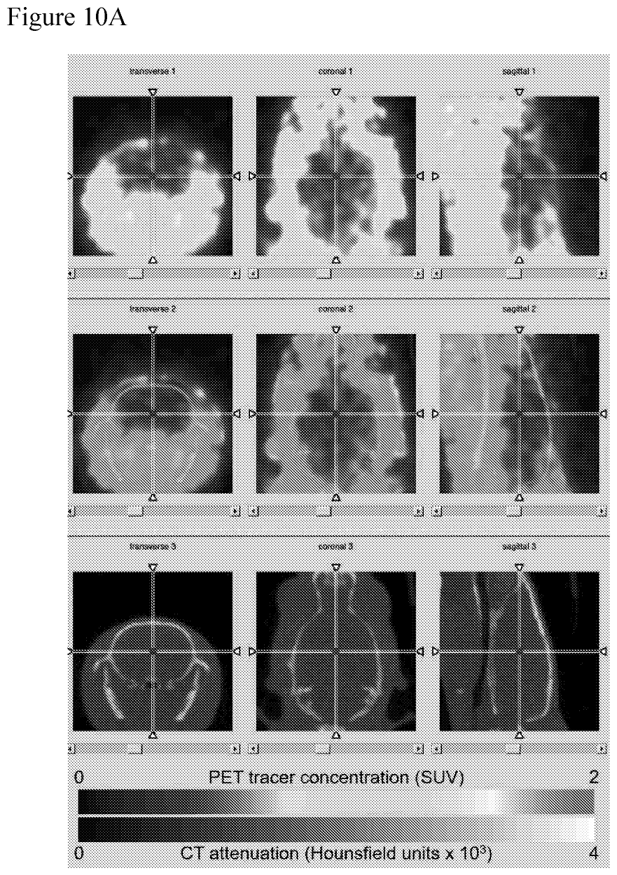

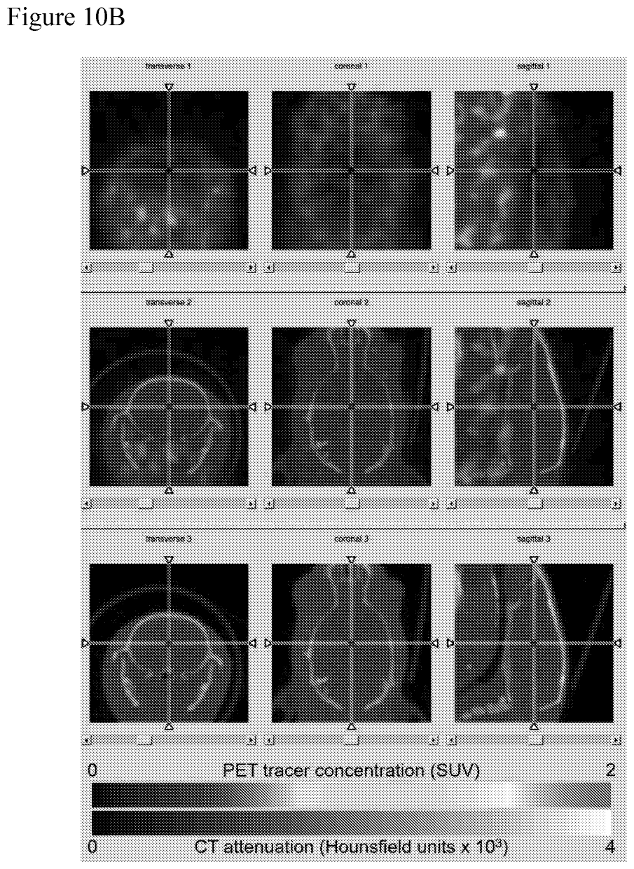

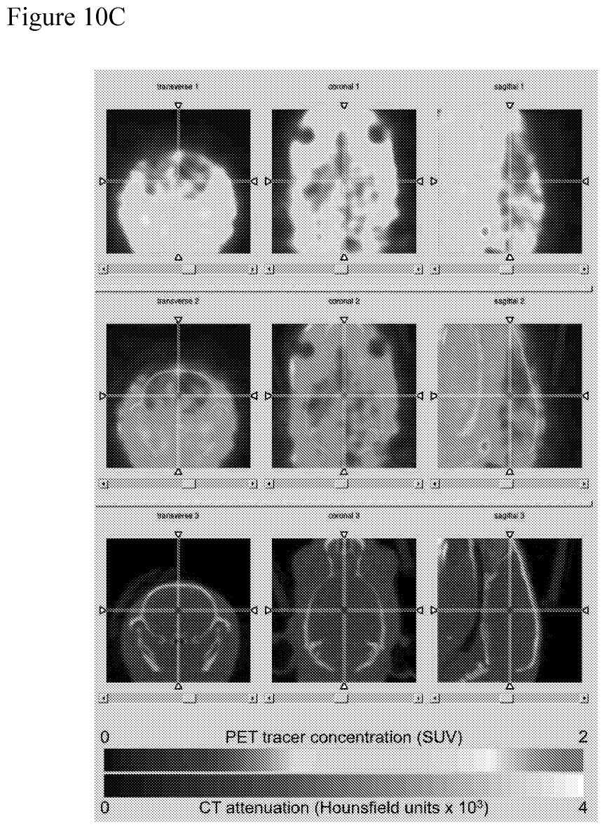

FIGS. 10A-10C show a MicroPET/CT image of a brain in a rat 10 minutes after administration of F-18-6FPBA (10A); 6-[¹⁸F]-fluorodopamine; and F-18-6FPBPE (10C).

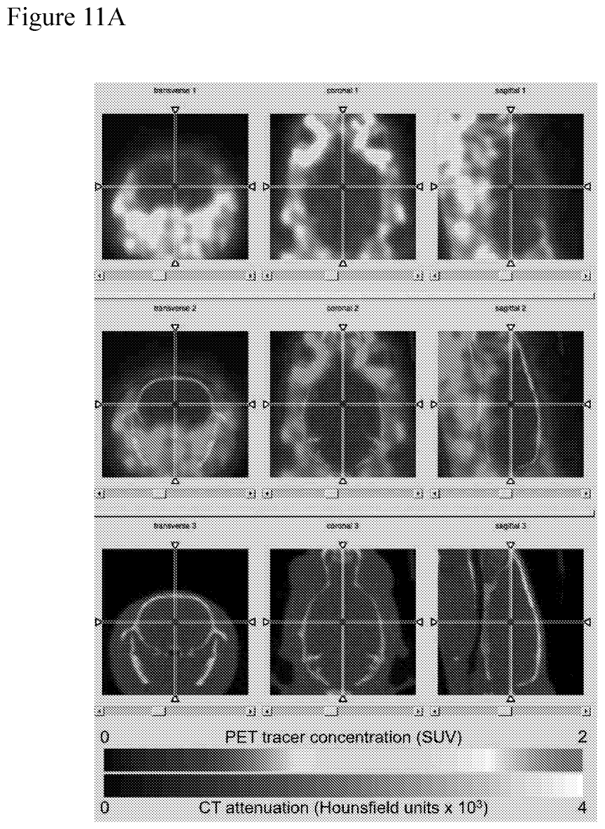

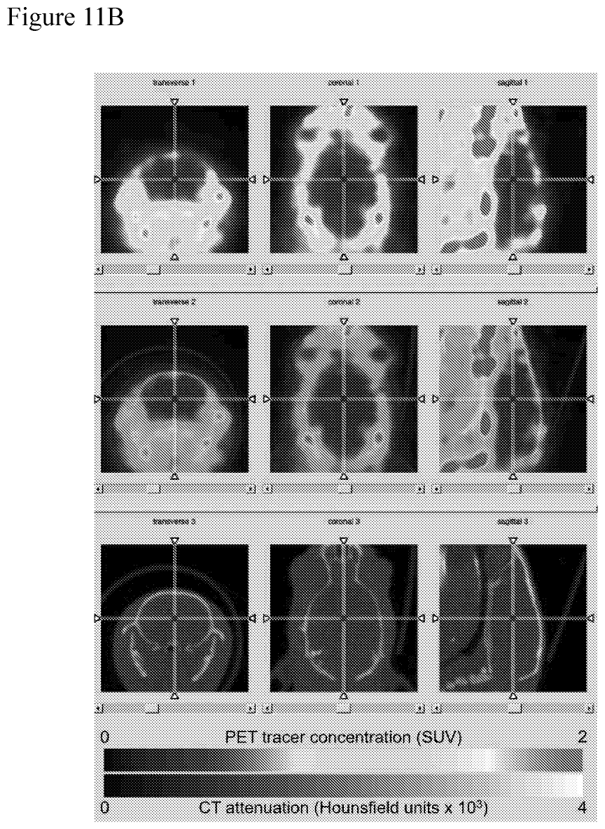

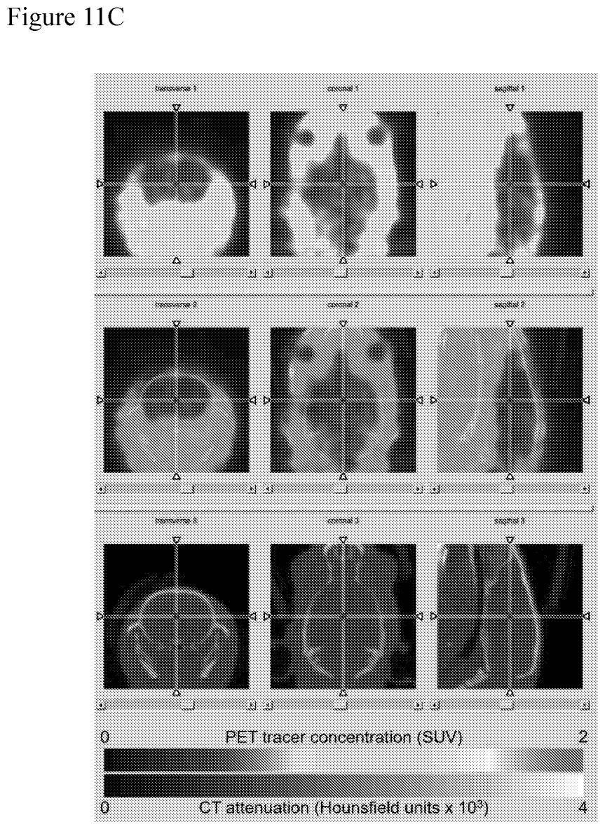

FIGS. 11A-11C show a MicroPET/CT image of a brain in a rat 60 minutes after administration of F-18-6FPBA (11A); 6-[¹⁸F]-fhrorodopamine (11B); and F-18-6FPBPE (11C).

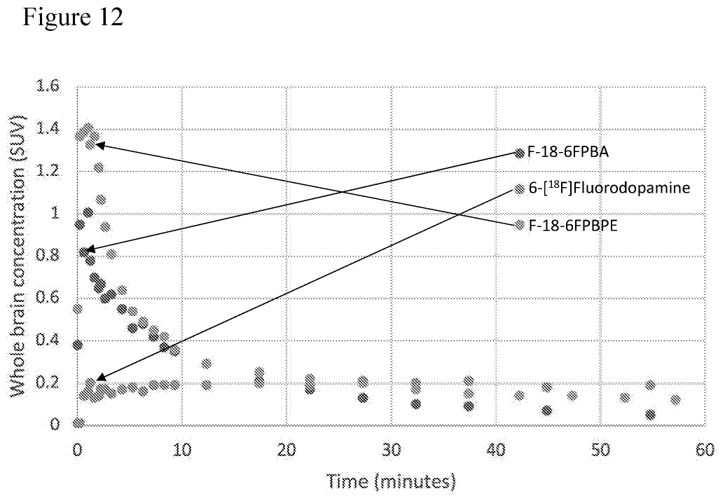

FIG. 12 shows a time activity curve a F-18-6FPBA, 6-[¹⁸F]-fluorodopamine, and F-18-6FPBPE.

As described herein, all embodiments or sub-combinations may be used in combination with all other embodiments or sub-combinations, unless mutually exclusive.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound selected from:

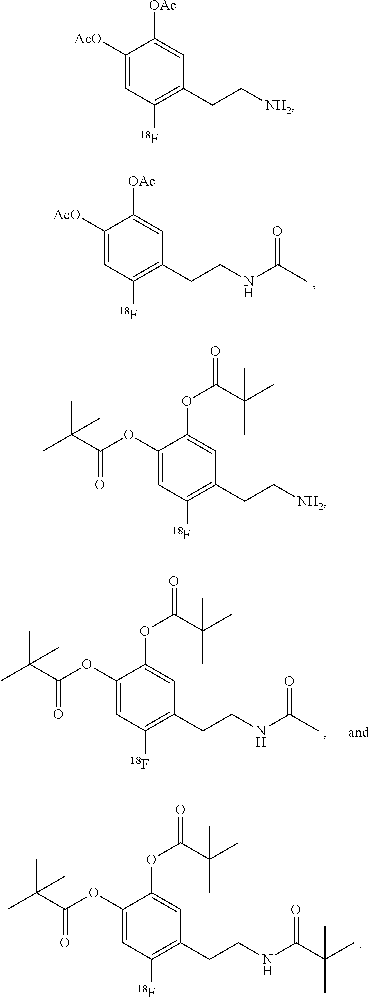

2. A compound selected from:

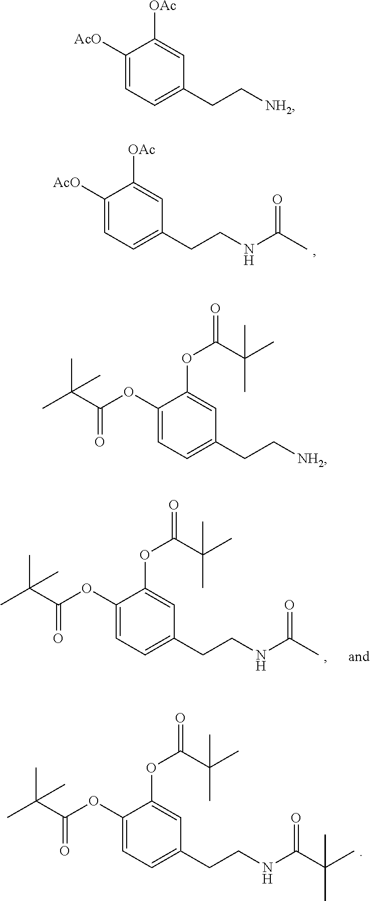

3. A pharmaceutical composition comprising a therapeutically or diagnostically effective amount of a compound or a pharmaceutically acceptable salt thereof and a pharmaceutically acceptable excipient or solvent,
wherein the compound is derived from a parent compound having a hydroxyl or amine moiety, and
wherein the hydroxyl in the parent compound is presented as an ester or a carbonate in the compound, or the amine in the parent compound is presented as an alkyl amine amide in the compound,
provided the parent compound is not morphine,
provided the parent compound is not 5,5′-((2-hydroxypropane-1,3-diyl)bis(oxy))bis(4-oxo-4H-chromene-2-carboxylic acid);
provided the compound is not heroin;
provided the compound is not (2<S,3R,4R,5<S,6R)-6-(acetoxymethyl)-3-(3-iodobenzamido)tetrahydro-2H-pyran-2,4,5-triyl triacetate;
provided the compound is not N-acetyl β-alanine; and
provided the compound is not acetylcholine.

4. The composition of claim 3, wherein the molecular weight of the compound is less than 450 Da, preferably less than 300 Da.

5. The composition of any one of claims 3-4, wherein the polar surface area of the compound is less than 100 Å², preferably less than 50 Å².

6. The composition of any one of claims 3-5, wherein the parent compound forms less than or equal to seven hydrogen bonds with water.

7. The composition of any one of claims 3-6, wherein the hydroxyl in the parent compound is presented as an ester or a carbonate in the compound.

8. The composition of any one of claims 3-6, wherein the amine in the parent compound is presented as a carbamate in the compound.

9. The composition of any one of claims 3-6, wherein the carboxylic acid present in the parent compound is presented as an ester in the compound.

10. The composition of any one of claims 3-6, wherein the parent compound is selected from a compound listed in Table 1, Table 2, or Table 3.

11. The composition of any one of claims 3-6, wherein the compound is selected from

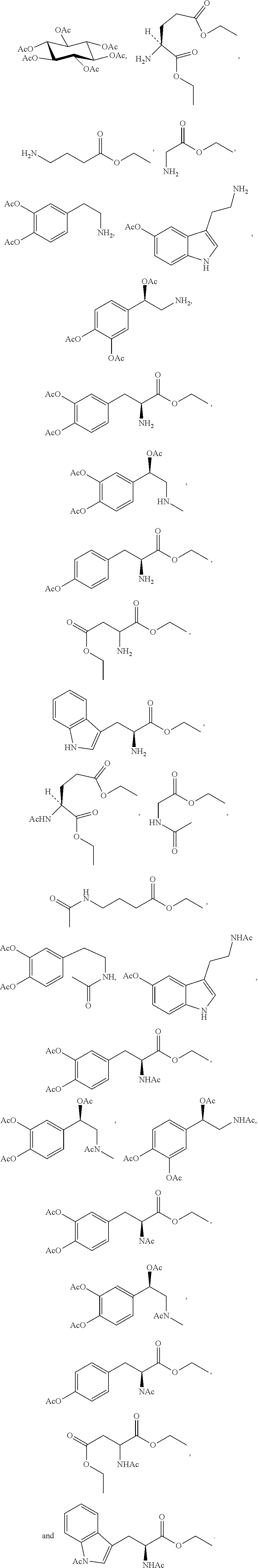

12. The composition of any one of claims 3-6, wherein the compound is selected from:

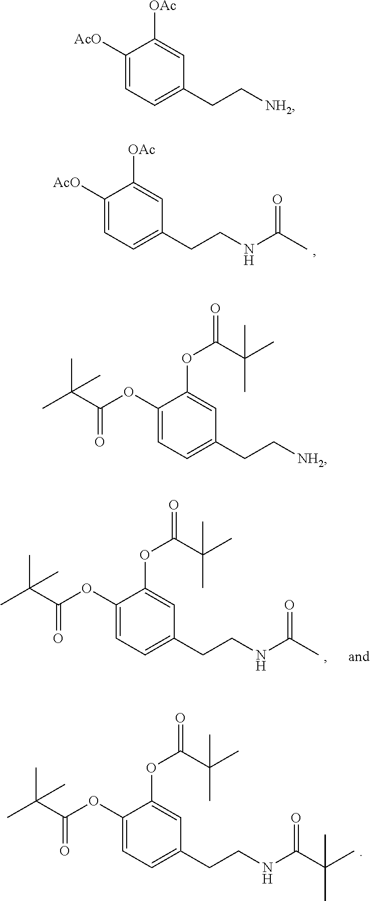

13. The composition of any one of claims 3-12, wherein the compound further comprises a radioimaging agent.

14. The composition of claim 13, wherein the radioimaging agent is a radionuclide.

15. The composition of claim 14, wherein the radioimaging agent is selected from ³H, ¹⁸F, ³⁶Cl, ⁷⁶Br, ⁷⁷Br, ¹²³I, ¹²⁴I, ¹²⁵I, and ¹³¹I.

16. The composition of claim 14, wherein the radioimaging agent is ¹⁸F.

17. The composition of any one of claims 3-6, wherein the compound is selected from:

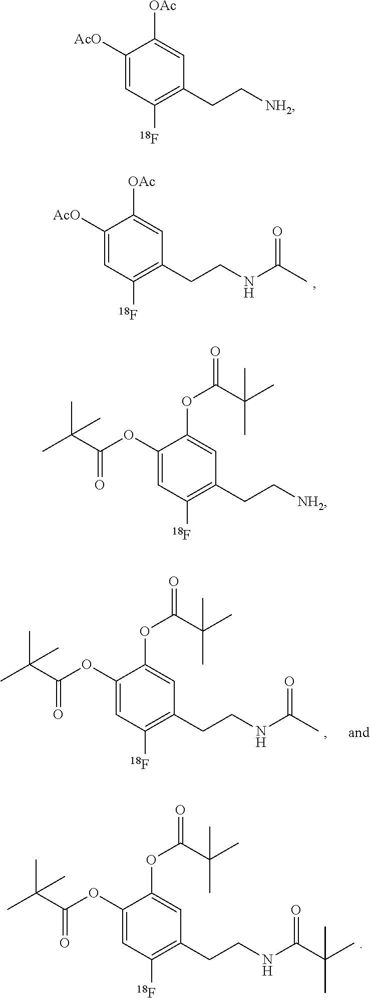

18. The composition of any one of claims 3-17, wherein the composition does not comprise mannitol.

19. The composition of any one of claims 3-18, wherein the therapeutically or diagnostically effective amount of the compound in the composition is less than 50% w/w of a therapeutically or diagnostically effective amount of the parent compound in a control composition to achieve the same therapeutic or diagnostic effect.

20. The composition of any preceding claim, wherein the excipient is a BBB disruptive excipient.

21. A transdermal patch comprising a pharmaceutical composition of any one of claims 3-20.

22. A method of treating or preventing a neurological disease in an organism, comprising administering to the organism a composition of any one of claims 3-20.

23. The method of claim 22, wherein the neurological disease is selected from Alzheimer's disease, neurofibromatosis, Huntington's disease, depression, amyotrophic lateral sclerosis, multiple sclerosis, stroke, Parkinson's disease, and dementia.

24. The method of claim 23, wherein the disease is Alzheimer's disease.

25. The method of claim 23, wherein the disease is Parkinson's disease.

26. The method of claim 23, wherein the disease is dystonia.

27. The method of claim 23, wherein the disease is neurological, mental or emotional related disease (e.g., depression).

28. The method of any one of claims 22-27, wherein the composition is administered dermally, orally, intravenously, or intraperitoneally

29. The method of claim 28, wherein the composition is administered dermally.

30. The method of any one of claims 22-29, wherein the organism is a mammal.

31. The method of claim 30, wherein the mammal is a human.

32. A method of treating or preventing cancer in an organism, comprising administering to the organism a composition of any one of claims 3-20.

33. The method of claim 32, wherein the cancer is cancer of the brain.

34. The method of claim 32 or 33, wherein the composition is administered dermally.

35. The method of any one of claims 32-34, wherein the organism is a mammal.

36. The method of claim 35, wherein the mammal is a human.

37. A method of imaging a brain of an organism suffering for a neurological disease comprising administering the composition of any one of claims 13-17.

38. The method of any one of claim 37, wherein the organism is a mammal.

39. The method of claim 38, wherein the mammal is a human.
